(19) United States
(12) Patent Application Publication
(10) Pub. No.: US 2022/0313749 A1
(43) Pub. Date: Oct. 6, 2022

(54) NOVEL LACTIC ACID BACTERIUM DERIVED FROM AMUR CORK LIVING IN SHIRAKAMI MOUNTAINS

(71) Applicants: HIROSAKI UNIVERSITY, Hirosaki-shi, Aomori (JP); LAVIEPRE CO., LTD., Hirosaki-shi, Aomori (JP)

(72) Inventors: Akio TONOUCHI, Aomori (JP); Hayato MAEDA, Aomori (JP); Kazuhide MIURA, Aomori (JP)

(21) Appl. No.: 17/597,012

(22) PCT Filed: Jul. 3, 2020
(86) PCT No.: PCT/JP2020/026131

§ 371 Date: Dec. 22, 2021

(30) Foreign Application Priority Data
Jul. 3, 2019 (JP) 2019-124232

(51) Int. Cl. A61K 35/744; A23K 10/18; A23L 33/135; C12N 1/20; A61P 3/04; A61P 3/06; A61P 3/10

(52) U.S. Cl. CPC A61K 35/744; A23K 10/18; A23L 33/135; C12N 1/20; A61P 3/04; A61P 3/06; A61P 3/10

(57) ABSTRACT

Novel lactic acid bacterium L8 strain and a composition including the same L8 strain, the composition having blood glucose level reducing action, a lipid metabolism improving action, an antioxidant action, an antiobesity action, a fatty liver suppressing action, a hepatitis preventing action, a liver function improving action, and an intestinal environment improving action. A Lactococcus lactis subsp. lactis L8 strain derived from Amur cork living in the Shirakami Mountains, a composition containing the strain, and a medium and a method for efficiently culturing the strain.

### TECHNICAL FIELD

The present invention relates to a novel Lactococcus lactis subsp. lactis L8 strain (hereinafter referred to as “L8 strain”), which is a novel lactic acid bacterium derived from Amur cork living in the Shirakami Mountains. Furthermore, the present invention relates to a composition containing the strain, and a medium and a method for efficiently culturing the strain.

### BACKGROUND ART

Due to the recent boom in nutritionally functional foods and the crisis of the insurance system, attention is focused on preventing illness without relying on pharmaceutical agents. In particular, functional foods and supplements containing lactic acid bacteria have been commercialized.

For example, Patent Literature 1 states that it was found that a Lactococcus lactis JCM5805 strain and the like have an interferon production-inducing action and it can be used in the form of pharmaceutical agents and foods as an immunostimulatory agent and a preventive/therapeutic agent for viral infections. In addition, Patent Literature 2 states that a Lactobacillus paracasei MCC1375 strain, a composition containing the strain, and a method of suppressing the onset of an influenza virus by administering the strain to promote the production of interleukin 12 and activate NK cells.

Lactic acid bacterium is a general term for a bacterium that produces a large amount of lactic acid from a saccharide such as glucose, and saccharide is required for culturing. Furthermore, nutrients such as a nitrogen source such as peptones are indispensable for culturing lactic acid bacterium, and a medium tends to be expensive. In Non-Patent Literatures 1 and 2, a medium for culturing lactic acid bacterium is studied.

The properties and functions of lactic acid bacterium vary depending on an isolation source and a strain, and in order to provide foods and supplements with more effective functions, a novel lactic acid bacterium strain and cultivating the strain efficiently and at lower cost are required.

### CITATION LIST

### Patent Literature

Patent Literature 1: WO 2012/091081
Patent Literature 2: WO 2012/133827

### Non Patent Literature

Non-Patent Literature 1: Zhao et al., AMB Expr, (2016), 6:65
Non-Patent Literature 2: C. Li et al., Journal of Biotechnology, 93, (2002), 27-34

### SUMMARY OF INVENTION

### Technical Problem

An object of the present invention is to provide a novel lactic acid bacterium L8 strain derived from Amur cork that lives in the Shirakami Mountains. An object of the present invention is also to provide a composition including the L8 strain, the composition having blood glucose level reducing action, a lipid metabolism improving action, an antioxidant action, an antiobesity action, a fatty liver suppressing action, a hepatitis preventing action, a liver function improving action, or an intestinal environment improving action. Furthermore, the present invention relates to a medium and a culturing method capable of culturing the L8 strain efficiently and at low cost.

### Solution to Problem

That is, in order to solve the above problems, the present invention includes the following embodiments.

### Embodiment 1

A Lactococcus lactis subsp. lactis L8 strain.

### Embodiment 2

A Lactococcus lactis subsp. lactis L8 strain isolated from Amur cork living in the Shirakami Mountains.

### Embodiment 3

A composition containing the strain according to (Embodiment 1) or (Embodiment 2).

### Embodiment 4

The composition according to (Embodiment 3), which has a blood glucose level reducing action, a lipid metabolism improving action, an antioxidant action, an antiobesity action, a fatty liver suppressing action, a hepatitis preventing action, a liver function improving action, or an intestinal environment improving action.

### Embodiment 5

The composition according to (Embodiment 3) or (Embodiment 4), which is a food, a supplement, a beverage, a pharmaceutical agent, or feed.

### Embodiment 6

A medium for efficiently culturing a Lactococcus lactis subsp. lactis L8 strain, the medium including (1) glucose, (2) ammonium sulfate, and (3) one or more kinds selected from a beef extract and a yeast extract, and no peptones.

### Embodiment 7

The medium according to (Embodiment 6), further including triammonium citrate and polysorbate 80.

### Embodiment 8

A method for culturing a Lactococcus lactis subsp. lactis L8 strain, the method including using a medium comprising (1) glucose, (2) ammonium sulfate, and (3) one or more kinds selected from a beef extract and a yeast extract, and no peptones.

### Embodiment 9

A blood glucose level lowering agent, a lipid metabolism improving agent, an antioxidant, an antiobesity agent, a fatty liver inhibitor, a hepatitis preventive agent, a liver function improving agent, or an intestinal environment improving agent containing the strain according to (Embodiment 1) or (Embodiment 2).

### Advantageous Effects of Invention

According to the present invention, it is possible to provide a novel lactic acid bacterium L8 strain derived from Amur cork that lives in the Shirakami Mountains, a composition including the L8 strain, the composition having blood glucose level reducing action, a lipid metabolism improving action, an antioxidant action, an antiobesity action, a fatty liver suppressing action, a hepatitis preventing action, a liver function improving action, or an intestinal environment improving action, and a medium and a culturing method capable of culturing the L8 strain efficiently and at low cost.

### DESCRIPTION OF EMBODIMENTS

Hereinafter, the present invention is described in detail.

Acquisition of L8 Strain

A sample (leaf) collected from the plant (Amur cork) living in the Shirakami Mountains was immersed in 10% skim milk medium and kept warm at 40° C. The culture in which the skim milk was solidified was streaked on an MRS agar medium containing 5% CaCO₃ and anaerobically cultured at 37° C. The Colony in which CaCO₃ was dissolved by lactic acid production and the surrounding became transparent was separated by streak culture again in the same medium.

The resulting strain was named Lactococcus lactis subsp. lactis L8.

Identification of L8 strain

Source of isolation: leaf of Amur cork

Strain: Lactococcus lactis subsp. lactis

Morphological features: Egg-shaped, 0.5-1.5 μm in size, gram stain positive, non-motile

Oxygen-related enzyme: oxidase/catalase negative

Growth temperature: Growth in the range of 10° C. to 40° C., optimum temperature is 37° C.

Lactobacillus: L-arabinose, D-ribose, D-xylose, D-galactose, D-glucose, D-fructose, D-mannose, D-mannose, N-acetylglucosamine, amygdalin, arbutin, salicin, D-cellobiose, D-maltose, D-lactose, D-sucrose, D-trehalose, gentiobiose, gluconate (among them, D-Glucose, N-Acetylglucosamine are good)

Non-lactic acid substrate: glycerol, erythritol, D-arabinose, L-xylose, D-adonitol, methyl-βD-xylopyranoside, L-Sorbose, L-rhamnose, dulcitol, inositol, D-sorbitol, Methyl-αD-mannopyranoside, methyl-αD-glucopyranoside, aesculin, D-melibiose, Inulin, D-melezitose, D-raffinose, starch, glycogen, xylitol, D-turanose, D-lyxose, D-tagatose, D-fucose, L-fucose, D-arabitol, L-arabitol, 2-ketogluconate, 5-ketogluconate.

Others: It has the coagulation action of skim milk and soy milk. Fermented liquor (23.5 mL) was recovered by a centrifugal force of 10,000×g per 40 g of coagulated soymilk.

As shown in Table 1, when the 16S rRNA gene partial sequence of the L8 strain was compared with the standard strain of a closely related species, it showed 100% homology with Lactococcus lactis subsp. lactis. It also showed relatively high homology with Lactococcus lactis subsp. hordniae, Lactococcus lactis subsp. tructae, and Lactococcus lactis subsp. cremoris. However, these are physiologically different from L8 because they cannot grow L8 or Lactococcus lactis subsp. lactis at 40° C. where they can grow. Therefore, L8 was identified as a new strain belonging to Lactococcus lactis subsp. lactis.

TABLE 1
Homology of 16S rRNA gene sequence of L8 strain and related species

| | Strain name | Accession | Homology |
|---|---|---|---|
| Scientific name | (Type strain) | number | ( % ) |
| Lactococcus lactis subs. lactis | JCM 5805 (T) | BALX01000047 | 100.00 |
| Lactococcus lactis subs. hordniae | NBRC 100931 (T) | BCVL01000058 | 99.81 |
| Lactococcus lactis subs. tructae | L105 (T) | EU770697 | 99.16 |
| Lactococcus lactis subs. cremoris | NCDO 607 (T) | AB100802 | 99.07 |
| Lactococcus taiwanensis | 0905C15 (T) | AB699722 | 98.51 |

The Shirakami Mountains is a general term for a vast mountainous area of approximately 130,000 ha that extends over the southwestern part of Aomori Prefecture and the northwestern part of Akita Prefecture. About 74% of the total area of the Shirakami Mountains belongs to Aomori Prefecture. The world's largest pristine beech forest is distributed, and it was registered as a World Heritage Site (natural heritage) in December 1993.

The composition of the present invention comprises an L8 strain. The L8 strain may be a viable bacterial cell or dead bacterial cell, may be liquid or powdery, and may further contain a culture of the strain. In addition to the bacterial cell, the culture includes a disrupted product of the bacterial cell, a freeze-dried product, a culture solution, a culture solution extract, and the like.

The compositions of the present invention can be a food, a supplement, a pharmaceutical agent, a beverage, or feed. Examples of the food include milk, a dairy product, a seasoning, a processed agricultural and forestry food, confectionery, a bread, grain flour, a noodle, a processed marine product, a processed livestock product, a fat and oil, a processed fat and oil, a cooked frozen food, a retort food, and a ready-to-eat food, and a food material.

When the composition of the present invention is provided as the supplement or the pharmaceutical agent, the shape thereof can be in the form of a tablet, a capsule, a jelly, a paste, a powder, a granule, a liquid and the like.

When the composition of the present invention is provided as the beverage, it can be in the form of a lactic acid bacterium beverage, a yogurt drink, a soft drink and the like.

When the composition of the present invention is provided as the feed, it can be in the form of powder form, pellet form, crumbled form, granule form, flake form, bulky form, milk replacer or the like.

The composition of the present invention may contain additives such as an excipient, a disintegrant, a binder, or a lubricant that do not interfere with the action of the L8 strain and are acceptable as the food, the supplement, the beverage, the pharmaceutical agent, or the feed. Examples of such an excipient include glucose, lactose, corn starch, and sorbitol. Examples of the disintegrant include starch, sodium alginate, gelatin powder, calcium carbonate, calcium citrate, and dextrin. Examples of the binder include dimethyl cellulose, polyvinyl alcohol, polyvinyl ether, methyl cellulose, ethyl cellulose, gum arabic, gelatin, hydroxypropyl cellulose, and polyvinylpyrrolidone. Examples of the lubricant include talc, magnesium stearate, polyethylene glycol, cured vegetable oil.

In addition to the excipient, the compositions of the present invention further include a mineral, a vitamin, flavonoids, polyphenols, an amino acid, a nucleic acid, an essential fatty acid, a refreshing agent, a sweetener, a colorant, a flavor, a stabilizer, and a preservative, a sustained release modifier, a surfactant, a solubilizer, or a wetting agent.

The L8 strain of the present invention has a blood glucose level reducing action, a lipid metabolism improving action, an antioxidant action, an antiobesity action due to these actions, and an intestinal environment improving action. Therefore, the composition containing the L8 strain has a blood glucose level reducing action, a lipid metabolism improving action, an antioxidant action, an antiobesity action, and an intestinal environment improving action. In addition, the L8 strain of the present invention has fatty liver suppressing action, a hepatitis preventing action, and a liver function improving action due to these actions. Therefore, the composition containing the L8 strain has a fatty liver suppressing action, a hepatitis preventing action, and a liver function improving action.

In the composition of the present invention, the content of the L8 strain is not particularly limited, but can be 0.01 to 99% by weight, preferably 0.5 to 50% by weight, and more preferably 1 to 30% by weight, based on the weight of the lactic acid bacteria cells, based on the total weight of the composition.

In one aspect, the present invention provides a blood glucose level lowering agent, a lipid metabolism improving agent, an antioxidant, an antiobesity agent, a fatty liver inhibitor, a hepatitis preventive agent, a liver function improving agent, and an intestinal environment improving agent, containing an L8 strain.

A medium for efficiently culturing the L8 strain of the present invention includes (1) glucose, (2) ammonium sulfate, and (3) one or more kinds selected from a beef extract and a yeast extract, and no peptones. Lactic acid bacterium is a general term for a bacterium that produces a large amount of lactic acid from a saccharide such as glucose, and saccharide is required for culturing. Furthermore, since nutrients such as peptones such as proteose peptone are essential as a nitrogen source, the medium tends to be expensive. When ammonium sulfate as the nitrogen source instead of peptones is used only in the presence of a beef extract or a yeast extract, which is a vitamin source, the present inventors found that it was possible to provide a medium with the same high culture efficiency as when peptones were used.

Furthermore, in the medium of the present invention, the content ratio (1):(2):(3) of (1) glucose, (2) ammonium sulfate, and (3) one or more kinds selected from a beef extract and a yeast extract are 0.5 to 2.0:0 to 1.0:1 to 2.5. From the viewpoint of culture efficiency and cost, 0.5:0.5:2.0 to 2.5 is preferable.

The medium of the present invention may further contain triammonium citrate and polysorbate 80.

In the MRS medium generally used for culturing lactic acid bacteria (see Table 2 for composition), the present inventors found that doubling the contents of triammonium citrate, polysorbate 80, and beef extract to the amounts used in the MRS medium can provide a medium with the same culture efficiency as the MRS medium, when all of sodium acetate, magnesium sulfate, manganese (II) sulfate, and dipotassium hydrogen phosphate are not contained.

That is, it was found that the medium has the same high culture efficiency as the MRS medium by adding 0.4 parts by weight of triammonium citrate, 0.2 parts by weight of polysorbate 80, and 2 parts by weight of beef extract to include about 0.2 to 0.5 parts by weight of any one or more kinds of sodium acetate and dipotassium hydrogen phosphate in the medium.

Furthermore, in the medium of the present invention, the content (part by weight) of each component is, for example,

glucose: 0.5,

ammonium sulfate: 0.5,

one or more kinds selected from beef extract and yeast extract: 2.0 to 2.5,

triammonium citrate: 0.4,

polysorbate 80:0.2, and

one or more kinds selected from sodium acetate and dipotassium hydrogen phosphate: 0.2 to 0.5,

but is not limited to this.

The present invention further relates to a method for culturing a Lactococcus lactis subsp. lactis L8 strain, the method including using a medium comprising (1) glucose, (2) ammonium sulfate, and (3) one or more kinds selected from a beef extract and a yeast extract, and no peptones.

The L8 strain can be cultured efficiently and at low cost by such a method.

### EXAMPLES

The present invention is described below with reference to specific embodiments, but the present invention is not limited to the embodiments, and it is appreciated that various changes and modifications thereof may be made by those skilled in the art without departing from the scope or intent of the invention as set forth in the appended claims.

### Test Example 1: Functional Evaluation of L8 Strain Using Mice

The effects of improving antiobesity and the intestinal environment by intaking dry powder of lactic acid bacterium L8 strain collected from Amur cork in the Shirakami Mountains were investigated.

Normal mouse C57BL/6J mice (male, 4 weeks old, 6 mice in each group) purchased from Charles River Laboratories, Japan were prepared. The control group was allowed to freely ingest drinking water, and the lactic acid bacterium-administered group was allowed to freely ingest lactic acid bacteria dry powder dissolved in drinking water at a concentration of 0.35 μg/mL. The feed was a free intake of a high-fat feed containing 20% lipid. After 15 weeks of breeding, the feed intake amount, water intake amount, body weight, blood glucose level, feces weight, white adipocyte weight, liver lipid weight, and serum GPT (ALT) level of each group were measured. The results are shown in FIGS. 1 to 7.

Although the feed intake amount and the water intake amount were the same in each group, the body weight, blood glucose level, and white adipocyte weight tended to decrease in the lactic acid bacterium group as compared with the control group. In addition, the lipid weight of the liver also showed a tendency to decrease, and the serum GPT (ALT) level, which is an index of liver inflammation, decreased. From the above, it was suggested that the lactic acid bacterium L8 strain has a blood glucose level reducing action, a lipid metabolism improving action, an antiobesity action, a fatty liver suppressing action, a hepatitis preventing action and the like. In addition, the feces weight of the lactic acid bacterium group showed a tendency to increase as compared with the control group. From the above, it was suggested that the lactic acid bacterium L8 strain induces an increase in good bacteria in the intestinal tract and has an antiobesity action, a fatty liver suppressing action, a hepatitis preventing action, and an intestinal environment improving action.

### Test Example 2: Examination of Medium for Culturing L8 Strain

Preparation of Medium

Using the MRS medium (the composition is shown in Table 2) as the basic medium, Reference Examples of the composition shown in Table 3 and eight types of media of Examples 1 to 7 were prepared. The Reference Example is a modified MRS medium.

TABLE 2
Composition of MRS medium (Unit: parts by weight)

| Component | MRS medium |
|---|---|
| Glucose | 2 |
| Proteose peptone | 1 |
| Yeast extract | 0.5 |
| Beef extract | 1 |
| Triammonium citrate | 0.2 |
| Polysorbate 80 | 0.1 |
| Sodium acetate | 0.5 |
| Magnesium sulfate | 0.01 |
| Manganese (II) sulfate | 0.005 |
| Dipotassium hydrogen phosphate | 0.2 |

Culture of L8 strain

We take 100 μL of preculture solution (2.5×10⁹ cells/mL) in which the Lactococcus lactis subsp. lactis L8 strain was cultured in MRS medium (manufactured by DIFCO) at 37° C. for 48 hours, and inoculated into 10 mL of each medium and counted the number of cells after standing culture at 37° C. for 48 hours. The results are shown in Table 3.

TABLE 3
(Unit: g/100 mL)

| | Reference | Examples | | | | | | |
|---|---|---|---|---|---|---|---|---|
| Component | Example | 1 | 2 | 3 | 4 | 5 | 6 | 7 |
| Glucose | 0.5 | 0.5 | 0.5 | 0.5 | 0.5 | 0.5 | 0.5 | 0.5 |
| Ammonium | 0 | 0.5 | 0.5 | 0.5 | 0.5 | 0.75 | 0.5 | 0.5 |
| sulfate | | | | | | | | |
| Proteose | 1 | 0 | 0 | 0 | 0 | 0 | 0 | 0 |
| peptone | | | | | | | | |
| Yeast extract | 0.5 | 0 | 0.5 | 0.5 | 0.5 | 0.5 | 0 | 0 |
| Beef extract | 1 | 2 | 2 | 2 | 2 | 2 | 2 | 1 |
| Triammonium | 0.2 | 0.4 | 0.4 | 0.4 | 0.4 | 0.4 | 0.4 | 0.4 |
| citrate | | | | | | | | |
| Polysorbate 80 | 0.1 | 0.2 | 0.2 | 0.2 | 0.2 | 0.2 | 0.2 | 0.2 |
| Sodium acetate | 0.5 | 0 | 0 | 0.5 | 0 | 0 | 0 | 0 |
| Magnesium | 0.01 | 0 | 0 | 0 | 0 | 0 | 0 | 0 |
| sulfate | | | | | | | | |
| Manganese (II) | 0.005 | 0 | 0 | 0 | 0 | 0 | 0 | 0 |
| sulfate | | | | | | | | |
| Dipotassium | 0.2 | 0 | 0 | 0 | 0.2 | 0 | 0.2 | 0.2 |
| hydrogen | | | | | | | | |
| phosphate | | | | | | | | |
| OD600 | 3.07 | 2.65 | 2.72 | 3.00 | 3.26 | 2.70 | 3.00 | 2.28 |
| (Average of 3 | | | | | | | | |
| consecutive | | | | | | | | |
| times) | | | | | | | | |
| Ratio of Base- | 1 | 0.86 | 0.89 | 0.98 | 1.06 | 0.88 | 0.98 | 0.74 |
| M and OD | | | | | | | | |
| Cell density | 4119240506 | 3555696203 | 3649620253 | 4025316456 | 4374177215 | 3622784810 | 4025316456 | 3059240506 |
| (cells/ml) | 4.12E + 09 | 3.56E + 09 | 3.65E + 09 | 4.03E + 09 | 4.37E + 09 | 3.62E + 09 | 4.03E + 09 | 3.06E + 09 |

### Test Example 3: Examination of Medium Cost

For each medium used in Test Example 2, the medium cost was calculated when Reference Example was 100. The second decimal place was rounded off in the calculation. The results are shown in Table 4.

TABLE 4

| | Reference | Example | Example | Example | Example | Example | Example | Example |
|---|---|---|---|---|---|---|---|---|
| | Example | 1 | 2 | 3 | 4 | 5 | 6 | 7 |
| Medium | 100 | 55.5 | 63.2 | 64.9 | 64.2 | 63.8 | 56.5 | 32.4 |
| cost | | | | | | | | |

From the above, it can be seen that Examples 3, 4, and 6 can efficiently culture the L8 strain at low cost.

### INDUSTRIAL APPLICABILITY

According to the present invention, it is possible to provide a novel plant-derived lactic acid bacterium having an effect of improving a blood glucose level reducing action, a lipid metabolism improving action, an antioxidant action, an antiobesity action, a fatty liver suppressing action, a hepatitis suppressing action, a liver function improving action, an intestinal environment improving action and being effective for maintaining health, as well as a food, a supplement, a beverage, a pharmaceutical agent, or a feed containing such lactic acid bacterium.

### BRIEF DESCRIPTION OF DRAWINGS

FIG. 1 is a graph showing the feed intake amount (a) and water intake amount (b) in the control group and the lactic acid bacterium-administered group in the functional evaluation of the L8 strain using mice.

FIG. 2 is a graph showing the results of measuring the body weights of the control group and the lactic acid bacterium-administered group in the functional evaluation of the L8 strain using mice.

FIG. 3 is a graph showing the results of measuring the blood glucose levels in the control group and the lactic acid bacterium-administered group in the functional evaluation of the L8 strain using mice.

FIG. 4 is a graph showing the results of measuring feces weights of the control group and the lactic acid bacterium-administered group in the functional evaluation of the L8 strain using mice.

FIG. 5 is a graph showing the results of measuring white adipocyte weights in the control group and the lactic acid bacterium-administered group in the functional evaluation of the L8 strain using mice.

FIG. 6 is a graph showing the results of measuring liver lipid weights in the control group and the lactic acid bacterium-administered group in the functional evaluation of the L8 strain using mice.

FIG. 7 is a graph showing the results of measuring GPT (ALT) values of serum, which is an index of hepatitis, in the control group and the lactic acid bacterium-administered group in the functional evaluation of the L8 strain using mice.

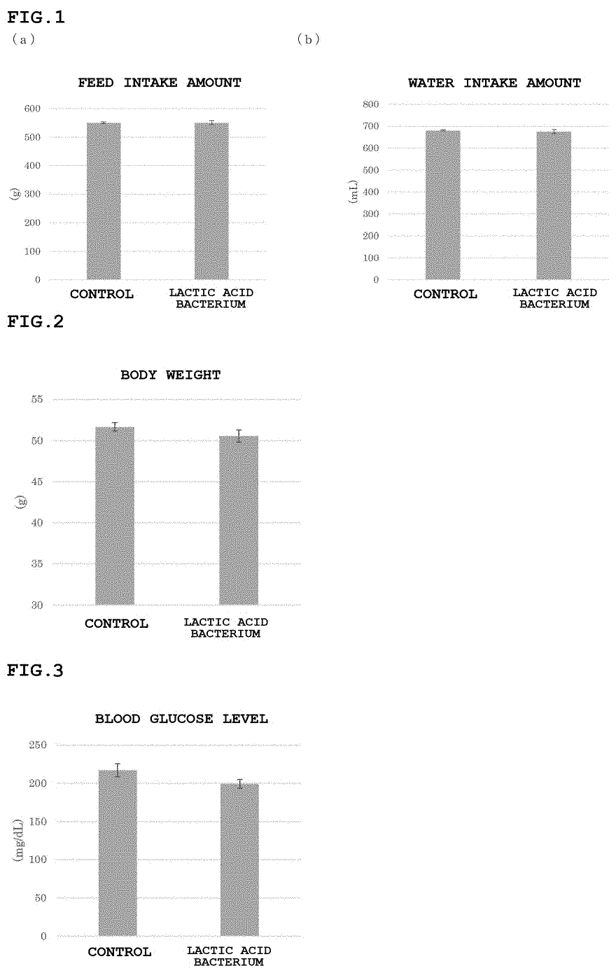

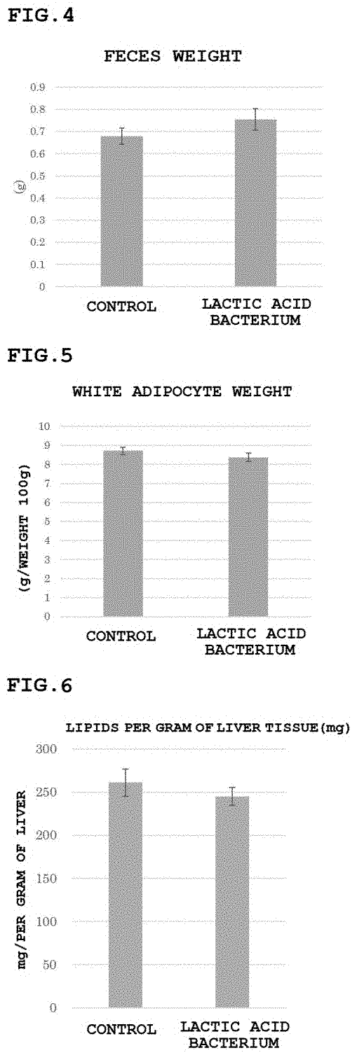

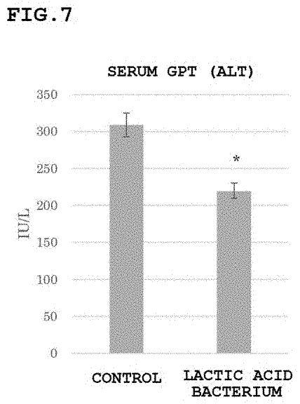

A Lactococcus lactis subsp. lactis L8 strain was deposited domestically on Jun. 19, 2019 at the National Institute of Technology and Evaluation NITE Patent Microorganisms Depositary (room 122, 2-5-8 Kazusakamatari, Kisarazu City, Chiba Prefecture 292-0818) under the identification label of “Lactococcus lactis 18” under the accession number NITE P-02970. After that, an application for transfer of the strain to an international deposit based on the Budapest Convention was submitted on Jun. 19, 2020, and a temporary number NITE ABP-02970 for the international deposit was assigned on Jun. 23, 2020. It is expected that deposit number (NITE BP-02970) of the international deposit will be assigned in the future.

1-2. (canceled)

3. A composition comprising:
a Lactococcus lactis subsp. lactis L8 strain isolated from Amur cork living in the Shirakami Mountains; and
at least one additive acceptable for a food, a supplement, a beverage, a pharmaceutical agent, or a feed.

4. The composition according to claim 3, which has at least one action selected from the group consisting of a blood glucose level reducing action, a lipid metabolism improving action, an antioxidant action, an antiobesity action, a fatty liver suppressing action, a hepatitis preventing action, a liver function improving action, and an intestinal environment improving action.

5. The composition according to claim 3, which is a food, a supplement, a beverage, a pharmaceutical agent, or a feed.

6. A medium for culturing a Lactococcus lactis subsp. lactis L8 strain, the medium comprising:
glucose;
ammonium sulfate; and
at least one material selected from the group consisting of a beef extract and a yeast extract,
wherein the composition includes no peptones.

7. The medium according to claim 6, further comprising triammonium citrate and polysorbate 80.

8. A method for culturing a Lactococcus lactis subsp. lactis L8 strain, the method comprising:
culturing the strain with a medium comprising:
glucose,
ammonium sulfate, and
at least one material selected from the group consisting of a beef extract and a yeast extract, and
wherein the medium includes no peptones.

9. The composition according to claim 5, wherein the composition is the pharmaceutical agent, which is a blood glucose level lowering agent, a lipid metabolism improving agent, an antioxidant, an antiobesity agent, a fatty liver inhibitor, a hepatitis preventive agent, a liver function improving agent, or an intestinal environment improving agent.

10. The method of according to claim 8, wherein the medium further comprising triammonium citrate and polysorbate 80.

11. A method of reducing a blood glucose level, the method comprising administering the composition according to claim 4 to a subject, wherein the composition has the blood glucose level reducing action.

12. A method of improving the lipid metabolism, the method comprising administering the composition according to claim 4 to a subject, wherein the composition has the lipid metabolism improving action.

13. A method of reducing oxidation in a body, the method comprising administering the composition according to claim 4 to a subject, wherein the composition has the antioxidant action.

14. A method of improving obesity, the method comprising administering the composition according to claim 4 to a subject, wherein the composition has the antiobesity action.

15. A method of suppressing a fatty liver, the method comprising administering the composition according to claim 4 to a subject, wherein the composition has the fatty liver suppressing action.

16. A method of improving a liver function, the method comprising administering the composition according to claim 4 to a subject, wherein the composition has the liver function improving action.

17. A method of improving an intestinal environment, the method comprising administering the composition according to claim 4 to a subject, wherein the composition has the intestinal environmental improving action.
